(19) United States
(12) Patent Application Publication
(10) Pub. No.: US 2012/0171658 A1
(43) Pub. Date: Jul. 5, 2012

(54) DEVICE AND PROCEDURE FOR THE MANUFACTURE OF BLOOD PRODUCTS

(75) Inventors: Gregor Bein, Giessen (DE); Holger Hackstein, Giessen (DE); Joachim Misterek, Wettenberg (DE)

(73) Assignee: JUSTUS-LIEBIG-UNIVERSITÄT GIESSEN, Giessen (DE)

(21) Appl. No.: 13/322,823

(22) PCT Filed: May 27, 2010
(86) PCT No.: PCT/EP2010/057344

§ 371 Date: Feb. 16, 2012

(30) Foreign Application Priority Data
May 27, 2009 (DE) 10 2009 022 793.8

(51) Int. Cl. B01D 17/00; C12N 5/078

(52) U.S. Cl. 435/2; 422/527

(57) ABSTRACT

The present invention relates to a device and a procedure for the withdrawal, manufacture, storage and transportation of blood products in the widest sense, in particular a closed blood removal, processing and storages system, as well as a system suitable for the administration of medicinal products which are obtained from blood or blood products, and a corresponding procedure for the legally conformant and GMP-compliant preparation, manufacture, storage and transportation of blood products or blood components or blood constituents or blood preparations (within the meaning of the Medical Products Act AMG). The present invention in particular allows the manufacture, storage, transportation and administration of completely aliquoted autologous serum eye drops for direct application to the patient or other drugs manufactured from blood or blood products without the need for clean room laboratories.

The present invention concerns a device and a procedure for the manufacture, storage, transportation and administration of blood products (or blood components or blood constituents or blood preparations within the meaning of the Medical Products Act AMG and the German Transfusion Law TFG) after withdrawal of blood, either as whole blood donation as well as by haemapheresis, without the need for clean room laboratories. In particular it relates to a closed blood collection and processing system as well as a system for the storage, the transportation and the administration of blood products and a corresponding legally conformant and GMP-compliant procedure for the withdrawal, manufacture, storage, transportation and administration of blood products without the need for clean room laboratories. In a very particular manner, the present invention relates to the manufacture, storage, transportation and administration of completely aliquoted blood products which are directly applicable to the patient like for example autologous serum eye drops or other drugs composed of blood products without the need for clean room laboratories.

The present invention thus in particular relates to a closed blood collection and processing system for the manufacture, storage, transportation and administration of autologous serum eye drops in a legally conformant and GMP-compliant manner for which no clean room laboratory is required.

Said device and said procedure can be applied to the withdrawal and processing of blood obtained from autologous or allogeneic blood donors, i.e. for blood of autologous or allogeneic origin. Thereby, if required, for blood of autologous or allogeneic origin, further means for sterilization may be provided at or in the device, e.g. in the form of liquids provided within said device.

Said device and said procedure may be utilized both after a whole blood donation as well as after haemapheresis.

Similarly, the device and the procedure are equally suited for human and animal patients, and in particular for vertebrates.

The present invention thus describes for the first time a possibility to manufacture drugs from autologous or allogeneic blood or from components thereof which can be aliquoted and directly administered to the patient by the patient himself or another person or to an animal, preferably a vertebrate, without further auxiliary tools.

This is of particular importance for patients with certain eye diseases, since the manufacture of autologous serum eye drops in a legally conformant and GMP-compliant manner is time-consuming and expensive, most of all due to the need for a clean room laboratory for the aliquoting and filling of autologous serum eye drops in suitable means of administration.

### STATE OF THE ART

The term “blood products” is understood to mean: Blood preparations within the meaning of the Medical Products Act, sera from blood within the meaning of the Medical Products Act as well as blood ingredients or blood components or blood preparations which are intended for the manufacture of active agents or drugs.

The term “blood donor” is understood to mean: A human or an animal, preferably a vertebrate, whom a certain amount of blood or blood components is collected from to be used as active agent or drug, or for the manufacture of active agents or drugs or other products which are intended for an application in humans or animals.

The term “blood” or “whole blood”, respectively, is understood to mean: Blood of a human or an animal, preferably a vertebrate, containing all native blood components, after a blood donation.

The term “blood components” is understood to mean: All components which can be derived from blood or whole blood, respectively, by manual or mechanical separation procedures or be produced directly by haemapheresis.

The term “haemapheresis” is understood to mean: The separation of blood or whole blood into various blood components using cell separators with extracorporeal circulation immediately at the blood donor, whereby the non-required blood components are immediately returned back to the donor.

The term “blood serum” or “serum” is understood to mean: Any part or the blood or whole blood, respectively, which after blood coagulation remains as liquid, protein-rich but cell-poor supernatant.

The term “own serum” or “autologous serum” is understood to mean: Own blood serum of the respective individual patient obtained after an autologous blood donation.

The term “eye drops” (oculoguttae) is understood to mean: A dosage form of medicinal products intended for use on or in the eye.

The term “autologous serum eye drops” is understood to mean: Eye drops which are produced from autologous serum.

The term “predonation sampling” is understood to mean: Removal of at least 15 ml of the initial blood volume of the blood donation in order to reduce bacterial contamination of the blood or whole blood during the blood collection procedure. In general, the predonation sampling is conducted using a predonation sampling bag.

The abbreviation “GMP” is understood to mean: good manufacturing practice guidelines followed during the manufacture of drugs, medicinal products, active agents as well as the manufacture of food and feed products.

The expert skilled in the art knows how to conduct legally conformant and GMP-compliant blood donation procedures, either as whole blood donation or also by haemapheresis (see e.g. German Transfusion Law TFG including Guidelines, or Annex 1 of EU Guidelines for good manufacturing practice). Similarly, the manufacture of blood components and blood products in a legally conformant and GMP-compliant manner is known to those skilled in the art. The same applies to the manufacture of eye drops. This applies to both human as well as veterinary medicine.

An important issue in the implementation of a blood collection and for the manufacture of blood products is the choice of a suitable container. Even though prior art knows numerous containers suitable to receive blood as disclosed in DE 1 065 138 A1 (Company. B. Braun, AT 15.6.1956) or U.S. Pat. No. 5,224,937 (NPBI, AT 21.06.1991), or also a system for blood withdrawal and blood collection as e.g. disclosed in DE 1 123 800 (Fenwal Laboratories, Inc., AT 12.2.1957). These however possess no suitable administration means allowing a direct, i.e. without auxiliary tools, administration of the drug generated from the blood product within the device to the patient, or the systems concerned are no closed systems in the sense that the system is not opened apart from the step of blood collection from the blood donor and the step of application to the patient.

The state of the art is therefore insufficient with respect to the availability of a suitable device for a simple, legally conformant and GMP-compliant manufacture, storage, transportation and most of all administration of blood products, in particular for eye drops produced from blood or blood serum.

In accordance with the regulations of the European Pharmacopoea (Pharmacopoea Europaea, Ph. Eur.), eye drops always have to be produced under sterile conditions. As pharmacy-only medication, these may in Germany only be dispensed in pharmacies. Eye drops are either available in single-dose vials made of plastic or in eye drop bottles made of special glass (amber Type I glass). In addition to industrial manufacturing, eye drops may also be prepared on prescription in pharmacies according to the respective formulation issued by the physician. For this purpose however, a clean room laboratory is required which is usually not available in pharmacies.

A particular problem is currently the manufacture, storage, transportation and administration of eye drops from blood serum in a legally conformant and GMP-compliant manner. These eye drops, especially in the form of autologous serum eye drops, play an important role in the treatment of eye diseases. Typical application fields of these eye drops fabricated from autologous serum are the treatment of dryness and irritation of the eye, glaucoma, inflammations of the conjunctiva (conjunctivitis) and cornea inflammations (keratitis).

Autologous serum is however meanwhile also applied successfully for other ophthalmological purposes. According to information presented in the annual report 2003 of the Rechts der Isar (Right hand side of the River Isar) Hospital of the TU Munich, as treatment of retinal hole formation in the region of greatest visual acuity (macula foramina), also gluing with autologous serum is meanwhile performed routinely, i.e. autologous serum is used as adhesive.

Other fields of application in which eye drops produced from serum of the donor himself (denoted as own or autologous serum) or from serum of other donors (denoted as allogeneic serum) proved to be successful in the last approximately 10 years are listed in “Eigenserum and alternative Blutprodukte zur Behandlung von Augenoberflächenerkrankungen” by G. Geerling et al. in “Der Ophthalmologe” 7, 2008, page 623ff and page 644ff, and concern a large number of eye diseases and ocular surface disorders.

At present, autologous serum eye drops are for example manufactured in England, where autologous blood donations are submitted to national clean room laboratories where they are processed into autologous serum eye drops, divided into aliquots, filled and subsequently returned to the attending physician. This procedure is time-consuming and expensive. A production of autologous serum eye drops in the blood establishment itself (e.g. blood bank) is so far impossible, since these establishments in the majority of cases possess none of the legally required clean room laboratories.

### AIM OF THE INVENTION

The aim of the present invention is therefore to overcome the aforementioned disadvantages of the prior art and to provide a device and a procedure in which and by which from blood of a blood donor medicinal products comprising blood or blood products in the widest sense like for example blood serum can be produced directly within said device and under continuous closure of said device (after filling and until administration) without the need of a clean room laboratory. The device thereby should be such that said drugs produced from blood or blood products can subsequently be applied directly from elements of said device and without further auxiliary means. In addition, said device and said procedure should be designed in such a way that the desired blood product can be produced as well from a whole blood donation as also by haemapheresis. The device and procedure should be designed in such a way that they are suitable for a use in human as well as in animal patients.

### SOLUTION

The aim is solved according to the present invention by a device according to claim 1. In particular, the device includes a sampling unit (1) and at least one collection and separation unit (5), which is characterized in that the device comprises at least one application unit (10), whereby sampling unit (1), collection and separation unit (5) and the at least one application unit (10) are connected with each other in fluid communication.

With respect to the procedure concerned, the aim is solved according to this invention by the subject matter of claim 15.

With the device described in the present invention and the procedure described herein, blood products such as for example serum or autologous serum eye drops or similar medicinal products obtained from blood or blood components can be produced, stored, transported and thus made available to the patients in a legally conformant and GMP-compliant manner within a short time and little use of personnel and material costs in each medical facility with a blood or autologous blood donation facility. This equally applies to whole blood donations as well as to haemaphereses, both for human as well as for animal patients.

The invention is characterized in that after the filling of the device with blood (preferably autologous blood), no further opening of the device takes place which would lead to conditions considered as unsterile (apart from the administration of the blood product manufactured according to this invention to the patient, like for example autologous blood serum eye drops when instilled in the eye). This means that the procedure of the present invention is designed such that a legally conformant and GMP-compliant (e.g. “Annex 1 of the EU Guidelines for good manufacturing practice” or “Gesetz zur Regelung des Transfusionswesens”, German transfusion law TFG) manufacture of blood products like for example said eye drops from the blood of allogeneic donors or autologous donors is ensured.

The device and procedure of the present invention therefore allow for the first time the direct manufacture of autologous serum eye drops at the location of the donation itself in compliance with all legal provisions.

It is immediately apparent to the expert in this field that the device according to this invention can be utilized with materials suitable for this purpose.

### EXAMPLES

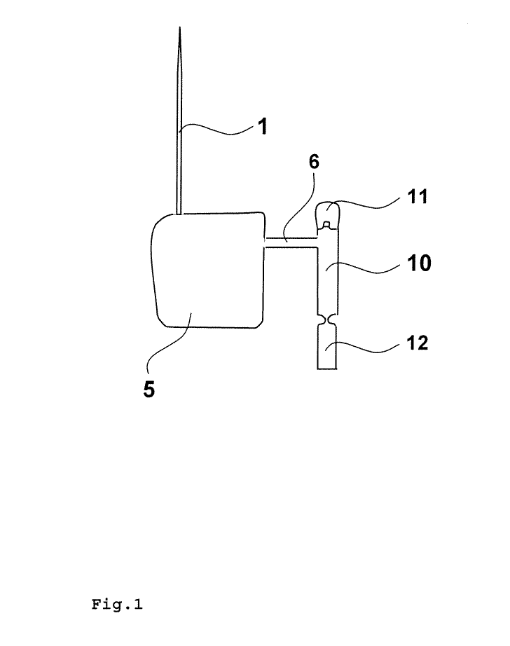

Embodiment examples are illustrated in the FIGS. 1-5:

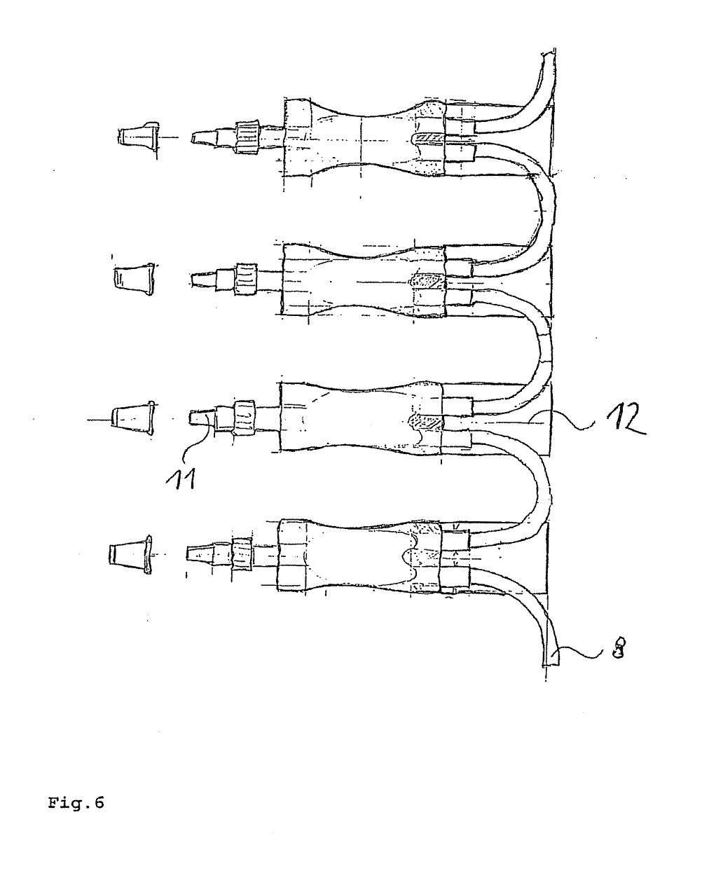

An additional embodiment example is illustrated in FIG. 6.

FIG. 1 shows the inventive device in a first embodiment in a sectional view with a sampling unit (1), a collection and separation unit (5), a feed line (6) and an application unit (10), which is provided in this embodiment with an opening means (11) and a means for a marking (12).

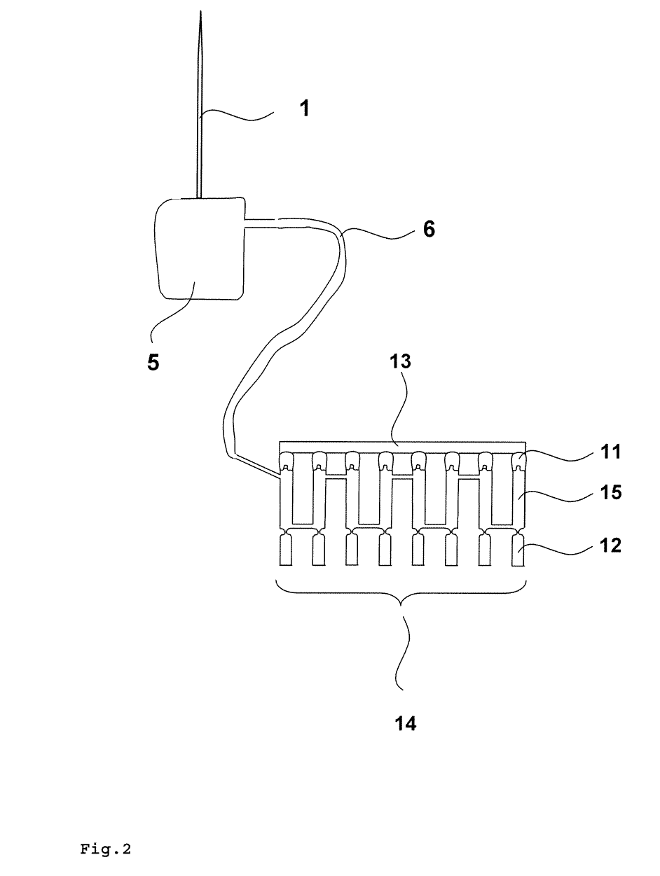

FIG. 2 shows a second embodiment—likewise in a sectional view—with a variety of application units (14), whereby the last application unit viewed in filling direction is denoted as the rearmost application unit (15).

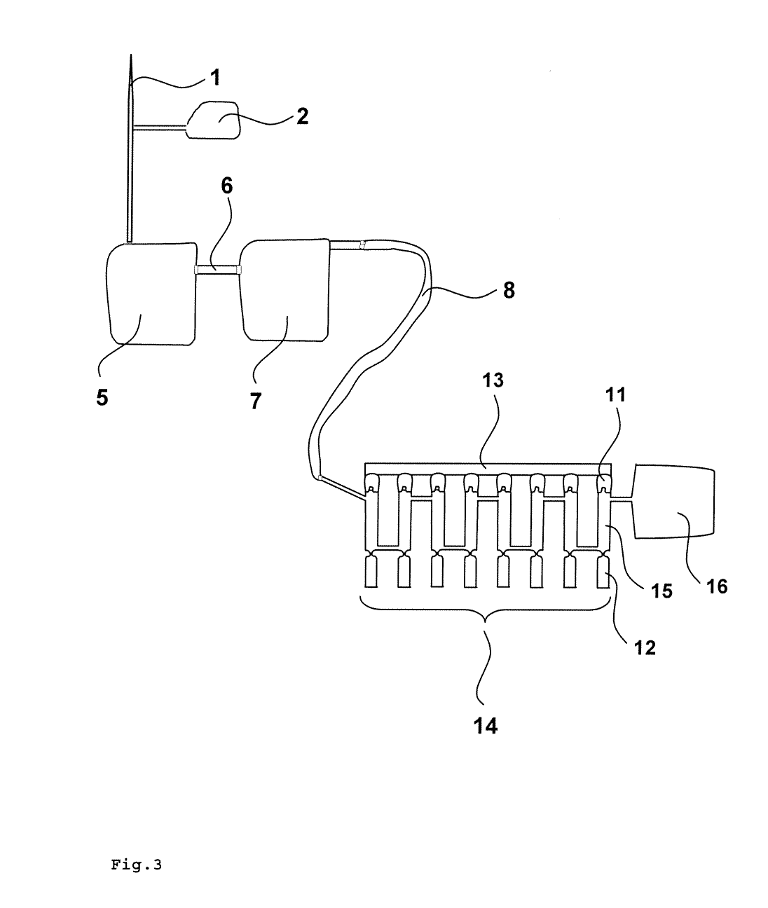

FIG. 3 shows a third embodiment—likewise in a sectional view—comprising a collection means to perform the predonation sampling (2), the so-called predonation sampling bag, an additional collection and separation unit (7), an additional feed line (8) and a terminal container (16).

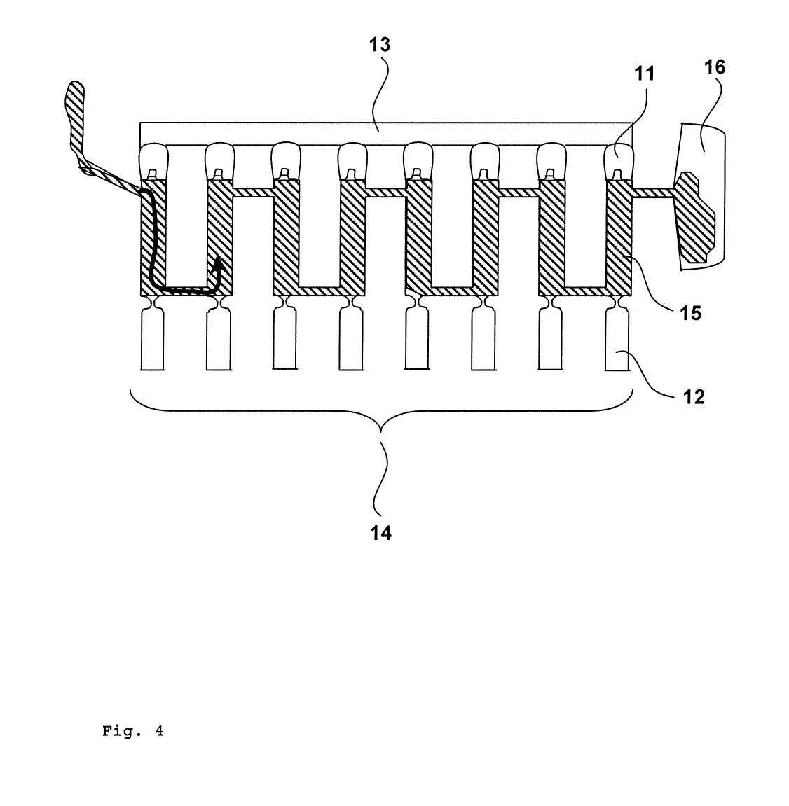

FIG. 4 shows—likewise in a sectional view—an enlarged view of the variety of application units (14) with a marking indicating the filling and the direction of filling.

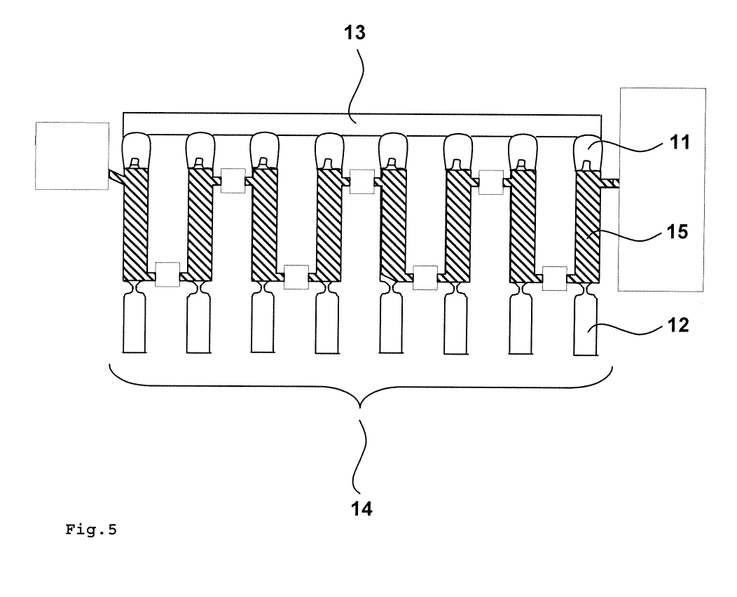

FIG. 5 shows—likewise in a cross-sectional view—an enlarged view of the variety of application units (14) after separation of the single application units from one another and the separation from the at least one feed line (6 or 8, respectively) and from the terminal container (16).

FIG. 6 shows an enlarged view of the variety of application units (14) which are depicted independently of the sampling, collection and separation units.

In the following, the figures are described in more detail:

FIG. 1 illustrates as the simplest embodiment the arrangement of the individual components of said device according to claim 1. The sampling unit (1) directs the collected blood into the at least one collection and separation unit (5) where, in the case that blood serum is desired as end product, blood coagulation takes place. The serum supernatant which results from the blood coagulation process is transferred from the at least one collection and separation unit (5) via the at least one feed line (6) into the at least one application unit (10). Preferably, the at least one application unit (10) is equipped with an opening means (11) which enables the person administering the medicinal product to open the application unit (10) prior to usage in a simple manner without further auxiliary equipment. Furthermore, the at least one application unit (10) is advantageously provided with a means for a marking (12) which allows to affix a permanent and unique identifier, for example an ongoing serial number or a barcode, to the application unit (10).

A further advantageous—not graphically illustrated—embodiment provides for the use of further agents (like for example an anticoagulants to obtain blood plasma, or for example a diluent for the dilution of the blood product obtained, or for example one or more pharmaceutically acceptable substances). Choice and arrangement of these further components are determined by the respective blood product desired and immediately apparent to the expert in this field.

Another particularly preferred—not graphically illustrated—embodiment example shows the provision of a barrier, for example in the form of a sling, a node, a clamp, a sphere, a cone, a valve, an encapsulated break-off part or a section intended to be bent and broken immediately in front of the discharge opening of the sampling unit (1) into the at least one collection and separation unit (5). To allow the influx of blood, the sphere, the cone, the encapsulated break-off part or the section intended to be bent and broken is then manually pushed into the at least one collection and separation unit (5) and retained here since the cross section of the feed line (6) is designed smaller than the cross section of the sampling unit (1). A similar barrier is preferably provided at the junction between the at least one collection and separation unit (5) and the feed line (6), in order to prevent the unintentional or untimely transfer of blood or blood components from the at least one collection and separation unit (5) into said feed line (6).

For those skilled in the art it is immediately apparent that the device according to this invention can be realized by the utilization of materials which are suitable and approved to be used for the collection, storage, transportation and administration of blood or blood products.

FIG. 2 illustrates in a more advantageous embodiment example the arrangement of the individual components of the device of this invention. The sampling unit (1) transfers the collected blood into the at least one collection and separation unit (5). The desired blood product is transferred from the at least one collection and separation unit (5) via the at least one feed line (6) into any number of application units (14). The eight application units depicted in FIGS. 2 to 5 are to be understood as purely exemplarily and should restrict on no account the possible number of application units. In the case that a variety of application units are used, these are arranged according to this invention in sequential order up to the rearmost application unit (15) and in such a way that they can be filled completely with the blood product intended for use in the patients. In addition, if a variety of application units is provided, these are according to this invention connected via a connecting bridge (13) to form one unit and can be removed individually.

In the case that a variety of application unit is provided, every single unit is also preferably equipped with an opening means (11) and a means for a marking (12).

Another particularly advantageous—not graphically depicted—embodiment example provides in addition the following modifications:
Provision of a branch (for example in the form of a three-way valve or a Y-connector) at the sampling unit (1), in order to divert the first approximately 15 ml of the blood donation in the sense of a predonation sampling into a separable part of the device, for example into a collection means to perform a predonation sampling (2), before the subsequently following blood is transferred into the at least one collection and separation unit (5) (see Guidelines of the German Transfusion Law). Said branch can particularly preferred be provided in the form of a so-called predonation sampling bag.
Parallel inflow to the variety of application units (14) through at least two feed lines, whereby the variety of application units (14) can be filled as well from below as also from above.

FIG. 3 illustrates in an even more advantageous embodiment the arrangement of the individual components of the device of the present invention. In addition to the aforementioned components, at least one additional collection and separation unit (7) is provided between the collection and separation unit (5) and the feed line (6) in order to improve the separation of the blood into the desired blood products. The collection means to conduct a predonation sampling (2) is attached to the sampling unit (1), which allows to obtain a blood sample for chemical, serological, laboratory diagnostic and bacterial examinations without compromising the sterility of the entire device. Particularly advantageous is the provision of a barrier, for example in the form of sling, a node, a clamp, a sphere, a cone, a valve, an encapsulated break-off part or a section intended to be bent and broken directly at the junction where the sampling unit (1) transitions into the collecting means to conduct the predonation sampling (2) (not shown in the drawing). Furthermore, a terminal container (16) is attached to the rearmost application unit (15) which facilitates the filling of the application units.

A further particularly preferred—not graphically depicted—embodiment example provides in addition the following modifications:
Provision of at least one container for the admixture of agents such as for example for the dilution of the manufactured blood product, the inhibition of blood coagulation, or the addition of further pharmaceutically acceptable substances.
Provision of a device to activate or deactivate at least once within the entire device of the present invention a stopping of the fluid flow in the direction of the flow path.
Provision of documentation means both before and after each separation line to ensure the assignment of the blood in the at least one collection and separation unit (5) to the at least one application unit (10 or 14, respectively).
Provision that the entire device is supplied in a sterile condition.
Provision that the entire device is supplied in a deflated state.
Provision that the entire device is supplied under excess pressure of sterile air to abolish the entry of non-sterile air during venous puncture in the blood collection process. In this case, providing a terminal container (16) for the uptake of air at the end of the flow path is mandatory.
Provision of a means allowing the passage of the blood or single blood products through a sterile filtration unit or a leukocyte filter or other cell filters.

FIG. 4 illustrates in enlarged detailed view the variety of application units (14) and the filling (indicated by hatched lines) of the application units with the desired blood product. The arrow indicates the flow direction of the blood product during the filling of the application units (14) according to the procedure of this invention. To achieve a complete filling of all application units, a sufficiently high quantity of the desired blood product is introduced into the application units to ensure that the flow of fluid reaches up to terminal container (16).

FIG. 5 illustrates in an enlarged detailed view the variety of application units (14) after separation of the connections in fluid communication between individual application units and separation from the at least one feed line (6 or 8, respectively) and the terminal container (16). The filling of application units with the desired blood product is indicated by hatched lines. At this point, application units filled with aliquoted fractions of the desired blood product are available for an application in the patient. Each application unit can individually be removed from the connecting bridge (13), opened via the opening means (11) and applied and utilized according to the present invention by the patient himself or another person.

Suitable materials for the device of this invention are for example, but not limited to:
polyvinyl chloride (PVC), polyurethane (PU, PUR)

Suitable sampling units are for example, but not limited to:
transfusion needles, hollow needles

Suitable agents provided in additional containers of the device which are connected with the units (1, 5 or 10) in fluid communication for the production of drugs from blood or blood products, are for example, but not limited to:
dilution solutions (for example saline solutions), anticoagulants (for example EDTA), further pharmaceutically acceptable substances (for example BSS®, Balanced Salt Solution)

Suitable separation means between the units (1, 5, 10) of the device are for example, but not limited to:
slings, nodes, clamps, spheres, scone, valves, encapsulated break-off parts, sections designed to be bent and broken, etc.

Suitable methods or tools for the sterile separation of plastic tubes or plastic hoses etc. are for example, but not limited to:
Separation welding, for example with squeezing/cutting tools like for example pliers
Thermal welding with predetermined tear line
Freezing of both ends beyond the separation line prior to the separation process
Ultrasonic welding

FIG. 6 illustrates in a detailed view a particularly advantageous arrangement of the variety of application units (14). Every single application unit has a screw top which is particularly preferred provided in the form of a luer lock screw cap with detachable cap. The afferent and efferent connections which are in fluid communication and located between the individual application units are configured as hose connections and arranged opposite of the respective screw caps to exclude possible injuries during the instillation of drops into the eye. The variety of application units is to be regarded independently of the sampling unit and the collection and separation unit. In a preferred embodiment, the various application units are each combined with appropriately sized collection and separation units by sterile welding so that, depending on the amount of serum eye drops needed, an appropriate amount of blood can be removed.

List of Single Process Steps:
1) After gaining access to the vascular system of a blood donor with the sampling unit (1), in particular by means of venous puncture which is known to those skilled in the art, blood is collected in at least one collection and separation unit (5).
Thereby, the following modifications are possible:
Addition of diluents, of agents to inhibit blood coagulation or of other pharmaceutically acceptable substances.
Provision of these agents within the device.
Sterile detachment of the sampling unit (1) from the rest of the device of the present invention after completion of blood collection.
Sterile detachment of the collection means intended for predonation sampling (2) after completion of blood collection from the rest of the device of the present invention.
2. Separation of the blood serum from the residual blood by coagulation and formation of the blood serum as supernatant above the residual blood components in at least one collection and separation unit (5).
Thereby, the following modifications are possible:
Blood coagulation at ambient temperature and for more than approx. 1 hour, preferably for more than 2 hours.
Particularly advantageous is to accelerate and to improve the separation of blood serum from residual blood components by centrifugation of the device according to this invention, at least however of the collection and separation unit (5).
3. Transfer of the blood serum obtained into the at least one application unit (10).
Thereby, the following modifications are possible:
If required, passage through a sterile filtration unit or a leukocyte filter or a cell filter.
Addition of further pharmaceutically acceptable substances or provision of further pharmaceutically acceptable substances within the device.
4. Sterile detachment of the at least one application unit (10) or the variety of application units (14), respectively, from the rest of the device of this invention. The application unit or units are thus now available for a use in the patient.

Further steps may optionally be added, if required, and comprise the aliquoting in the case that a variety of application units (14) is used, the control of sterility (which is for example facilitated by providing a collection means which allows to conduct a predonation sampling (2)), the storage of the application unit which is filled with the desired blood product, for example using frozen storage at approximately −20° C., or a storage for administration within 12 to 24 hours at 4° C. and discarding of the application dose in the case of non-utilization within the intended time period. A transport of the application unit filled with the desired blood product is equally possible.

### REFERENCE LIST

(1) Sampling unit

(2) Collecting means to perform predonation sampling

(5) Collection and separation unit

(6) Feed line

(7) Additional collection and separation unit

(8) Additional feed line

(10) Application unit

(11) Opening means

(12) Means for a marking

(13) Connecting bridge

(14) Variety of application units

(15) Rearmost application unit

(16) Terminal container

1. Device for the withdrawal of blood and the manufacture of blood products thereof and their storage and transportation, comprising sampling unit and at least one collection and separation unit, characterized in that said device includes at least an application unit, whereby sampling unit, collection and separation unit and the at least one application unit are connected in fluid communication.

2. Device according to claim 1, characterized in that sampling unit, collection and separation unit and the at least one application unit are made of sterilizable material like preferably polyvinyl chloride (PVC) or polyurethane (PU, PUR), and either designed in one piece or composed of several single elements.

3. Device according to claim 1, characterized in that sampling unit, collection and separation unit and the at least one application unit are designed such that a removal of at least one of the at least one application units from the rest of said device is possible without loss of sterility of said blood products contained within the device, at least however contained in said at least one application unit.

4. Device according to claims 1, characterized in that said at least one application unit has opening means which are designed in a suitable way to be opened by a patient or another person without further auxiliary equipment.

5. Device according to claims 1, characterized in that a variety of application units is provided.

6. Device according to claims 1, characterized in that said application units, respectively, have a volume of up to 400 ml per application unit.

7. Device according to claim 1, characterized in that between said collection and separation unit and said application units, an additional collection and separation unit is provided which is designed to be suitable for a separation of the blood components from each other and the further transport of the individual blood components.

8. Device according to claims 1, characterized in that at least the collection and separation unit or the entire device is designed in such a way and from such materials, preferably plastic like polyvinyl chloride (PVC) or polyurethane (PU, PUR), that a centrifugation of the content is possible without any loss of sterility of said content, i.e. basically and without damage to the walls of the collection and separation unit or the entire device.

9. Device according to claims 1, characterized in that if a variety of application units is used, these are either arranged in such a way to allow a serial inflow of fluids and then in alternating order each above and/or below in relation to the shape of the preferably extended application unit, or allow a parallel inflow of the contents of the collection and separation unit.

10. Device according to claims 1, characterized in that in flow direction during the delivery of the content from the collection and separation unit or the additional collection and separation unit into the at least one application unit—a terminal container is provided for the uptake of air and/or excessive content beyond this at least one application unit and connected with it in fluid communication, or, in the case of a variety of application units with serial inflow of fluids, a terminal container for the uptake of air and/or excessive content is provided after the—in flow direction—rearmost application unit.

11. Device according to claims 1, characterized in that at the sampling unit, a further collecting means is provided to carry out predonation sampling.

12. Device according to claims 1, characterized in that between the units of the device in fluid communication, at least however between the collection and separation unit and the at least one application unit, a means of separation are intended which can be activated or deactivated at least once.

13. Device according to claims 1, characterized in that adjacent to both sides of the provided separation line between sampling unit and the collecting means to conduct predonation sampling, as well as between collection and separation unit and the at least one application unit, each of said units is provided with at least one documentation field or is provided in already labeled condition in order to allow a subsequent assignment of the blood donor to the at least one application unit.

14. Device according to claims 1, characterized in that it is provided with agents in additional containers for the manufacture of a medicinal product from the blood of a donor, for example for the purpose of dilution, to prevent blood coagulation, or for the use of further pharmacologically acceptable substances which are connected in fluid communication with said units of the device, or said units are provided already prefilled with said agents.

15. Procedure for the withdrawal of blood and manufacture of blood components thereof in a device according to claim 1, characterized by the following steps:
1. Collection of the blood in the collection and separation unit.
2. Separation of the blood into at least one blood product and residual blood components in said collection and separation unit.
3. Transfer of the at least one blood product into the at least one application unit.
4. Sterile detachment of said at least one application unit from the rest of said device according to claim 1.

16. Procedure according to claim 15, characterized in that the at least one blood product is passed through a sterile filtration unit or a leukocyte filter or other cell filter.

17. Procedure according to claims 15, characterized in that the passage of said at least one blood product through a sterile filtration unit or a leukocyte filter or other cell filter is performed after the collection and separation unit.

18. Procedure according to claims 15, characterized in that the at least one blood product is serum and the separation of the blood is performed by blood coagulation and formation of the serum as supernatant above the residual components of the blood.

19. Procedure according to claims 15, characterized in that the separation of blood products is performed by centrifugation of said device according to claim 1 or said collection and separation unit.

20. Procedure according to claim 15, characterized in that after completion of blood withdrawal, said sampling unit is detached from the rest of the device of the present invention in a sterile manner.

21. The device according to claim 6 characterized in that said application units, respectively, have a volume of up to 100 ml per application unit.

22. The device according to claim 6 characterized in that said application units, respectively, have a volume of up to 10 ml per application unit.

23. The device according to claim 11 wherein the further collecting means is provided in the form of a predonation sampling bag.

24. The device according to claim 12 wherein the separation means are in the form of sections designed to be bent and broken.

25. The device according to claim 13 wherein the labeled condition is provided by a stamping at the wall of the units.

26. The device according to claim 13 wherein the labeled condition is in the form of an ongoing serial number.

27. The device according to claim 13 wherein, the labeled condition is in the form of a barcode.
